(19) United States
(12) Patent Application Publication
(10) Pub. No.: US 2026/0174460 A1
(43) Pub. Date: Jun. 25, 2026

(54) AUTO DISCONNECT TISSUE CUTTING DEVICE

(71) Applicant: Caldera Medical, Inc., Westlake Village, CA (US)

(72) Inventors: Joel Craig Martin, Westlake Village, CA (US); Ishan Kamal Khan, Westlake Village, CA (US)

(73) Assignee: Caldera Medical, Inc., Westlake Village, CA (US)

(21) Appl. No.: 19/427,492

(22) Filed: Dec. 19, 2025

Related U.S. Application Data
(60) Provisional application No. 63/736,496, filed on Dec. 19, 2024.

(51) Int. Cl. A61B 17/32; A61B 1/018; A61B 17/00; A61B 17/42

(52) U.S. Cl. CPC A61B 17/32002; A61B 1/018; A61B 17/42; A61B 2017/00017; A61B 2017/00398; A61B 2017/00477; A61B 2017/00544; A61B 2017/00561; A61B 2017/00734; A61B 2017/320024; A61B 2017/320028; A61B 2217/005

(57) ABSTRACT

A uterine tissue resection system may include a housing enclosing a power source such as a battery, a motor, and a control board for regulating power to the motor and the battery. Extending distally from the housing may be a cutting assembly comprising an outer cutting tube with an inner cutting tube received therein. The inner cutting tube may be fluidly coupled to an external suction source through a swivel connection interface. The housing may further contain a drive assembly connecting the inner cutting tube to a driveshaft of the motor so that rotation of the driveshaft results in simultaneous rotation and reciprocation of the inner cutting tube.

### FIELD OF THE INVENTION

The present invention relates to the field of uterine tissue resection, and more particularly, to systems, devices, or techniques for the intrauterine removal of abnormal tissues.

### BACKGROUND OF THE INVENTION

Uterine tissue resection devices are specialized instruments used in minimally invasive intrauterine procedures performed to remove various abnormal tissues, such as polyps, fibroids, or myomas. The use of such resection devices helps to limit potential complications and provide relatively short recovery times for a patient. In use, such resection devices are generally connected to both a hysteroscope to help enable precise positioning within a patient and an external suction source to help draw tissue in, and remove tissue through, the rotating and/or reciprocating tubular cutting elements used to resect tissue.

However, while relatively sophisticated in some respects, existing uterine tissue resection devices are still quite basic in other aspects and may thus possess several shortcomings. For example, existing devices generally rely on an external or otherwise separate power and control sources to power their rotating and/or reciprocating cutting elements. This may significantly increase the capital cost of such devices and limit their mobility, as such power sources are often relatively expensive and difficult to transport. Moreover, existing devices are generally fixedly connected to external suction sources or vacuum generators with suction tubing that may restrict the positioning of the tissue resection device during an operation, such as during rotational alignment of a cutting window of the device with abnormal target tissue.

Additionally, at least due to economic considerations, the rotating and/or reciprocating tubular cutting elements of existing uterine tissue resection devices generally utilize relatively basic design geometry (i.e. three-dimensional shapes, profiles, contours, etc.), which may limit the extent to which internal and/or external features of these cutting elements may be feasibly customized or optimized to improve cutting efficiency.

### SUMMARY OF THE INVENTION

In some examples, the techniques described herein relate to a uterine tissue resection system including: a housing including a handle; a power source such as a battery within the housing; a motor within the housing and connected to the battery; an outer cutting tube having a first lumen, a closed distal end, and a window through a sidewall thereof leading to the first lumen, the window being located proximal of the closed distal end; an inner cutting tube received within the first lumen and having a second lumen extending between an open distal end and an open proximal end, wherein the open proximal end is connected to an external suction source thorough a swivel interface adapted to enable free rotation of the housing relative to the external suction source; and a drive assembly located within the handle and connecting the inner cutting tube to a driveshaft of the motor such that when the motor receives current from the battery, rotation of the driveshaft causes the inner cutting tube to rotate and reciprocate concurrently.

In some examples, the techniques described herein relate to a system, wherein the swivel interface includes a first swivel connection fluidly coupling the external suction source to a suction tube.

In some examples, the techniques described herein relate to a system, wherein the first swivel connection includes a first rotatable connector including a first end including a barb nipple and an opposite second end including a barb nipple.

In some examples, the techniques described herein relate to a system, further comprising a second swivel connection fluidly coupling the suction tube to the inner cutting tube; and wherein the second swivel connection includes a second rotatable connector including a first end including a barb nipple and a second end adapted to receive a seal extending radially outward from a proximal end of the inner cutting tube.

In some examples, the techniques described herein relate to a system, further including a control board located within the housing, the control board adapted to regulate operation of the motor and discharging of the battery.

In some examples, the techniques described herein relate to a system, wherein the drive assembly includes: an inner cutting tube received within the outer cutting tube, wherein the inner cutting tube is adjacent to the driveshaft and parallel thereto; a first spur gear connected to the driveshaft; a second spur gear surrounding the inner cutting tube and meshed with the first spur gear; a worm gear positioned adjacent and meshed with a worm connected to the driveshaft such that rotation of the worm around a first axis results in rotation of the worm gear around a second axis; and a crank attached to the worm gear and a sleeve encompassing a portion of the inner cutting tube.

In some examples, the techniques described herein relate to a system, wherein the second spur gear defines a longitudinal groove and the inner cutting tube includes a sleeve defining a longitudinal projection; wherein the longitudinal groove is configured to receive the longitudinal projection to enable the second spur gear to rotate the inner cutting tube as the inner cutting tube reciprocates proximally and distally within the second spur gear.

In some examples, the techniques described herein relate to a system, wherein the second axis is perpendicular to the first axis.

In some examples, the techniques described herein relate to a system, further including: a power switch connected to the control board; and an activation switch connected to the control board, wherein the control board is adapted prevent the motor from receiving power from the battery unless the power switch and the activation switch are closed.

In some examples, the techniques described herein relate to a system, further including a safety switch connected to the control board, wherein the control board is adapted to provide power from the battery to the motor, after the activation switch is released, until the safety switch opens.

In some examples, the techniques described herein relate to a system, wherein a lever of the safety switch remains in continuous contact with a surface of the worm gear such that the safety switch is open when the inner cutting tube is in an extended position.

In some examples, the techniques described herein relate to a system, wherein the surface includes a semi-circular segment, which extends at least partially around a central axis of the worm gear, and a flattened segment; and wherein the safety switch is open when the lever is in contact with the flattened segment.

In some examples, the techniques described herein relate to a method of resecting intrauterine tissue using a uterine tissue resection system, the method including: grasping a handle of the uterine tissue resection system; introducing a cutting assembly into a target area, the cutting assembly extending from the handle and having an outer cutting tube with a closed distal end and a first lumen extending proximally from the closed distal end, wherein the first lumen carries an inner cutting tube attached to a drive assembly within the handle that is adapted to cause rotation and reciprocation of the inner cutting tube within the outer cutting tube; activating a suction source connected to the tissue resection system tissue to continuously apply suction to a second lumen extending through the inner cutting tube; and activating a motor within the handle to cause the inner cutting tube to rotate while translating from a retracted position in which the cutting window is unobstructed to an extended position in which the inner cutting tube closes the cutting window to cut tissue positioned within the cutting window, wherein activating the motor includes regulating power from a battery located within the handle to the motor via a control board located within the handle.

In some examples, the techniques described herein relate to a method, wherein the method includes rotating the handle of the uterine tissue resection system relative to an external suction source to cause a suction tube located within the handle to freely rotate with respect to the suction source.

In some examples, the techniques described herein relate to a method, wherein causing the suction tube within the handle to freely rotate with respect to a tube of the suction source includes rotating a first rotatable connector engaged with a proximal end of the suction tube and concurrently rotating a second rotatable connector engaged with a distal end of the suction tube.

In some examples, the techniques described herein relate to a method, further including stopping suction to the cutting window after a desired amount of tissue has been resected, and wherein stopping suction to the cutting window comprises placing the inner cutting tube in the extended position to block the cutting window.

In some examples, the techniques described herein relate to a method, wherein placing the inner cutting tube in the extended position comprises opening an activation switch supplying current to a motor through the control board, resulting in the control board enabling all power to the motor to flow through a safety switch that opens only when the inner cutting tube is in the extended position.

In some examples the techniques described herein relate to a method, wherein opening the safety switch includes rotating a gear of the drive assembly that is in continuous contact with the safety switch into a position where a lever of the safety switch is in contact with an insert segment of the gear.

In some examples, the techniques described herein relate to a method, wherein activating the motor within the handle includes closing the activation switch and a power switch electrically connected to the control board.

In some examples, the techniques described herein relate to a method, wherein introducing the cutting assembly into the target area includes inserting the cutting assembly through a working channel of a hysteroscope.

### BRIEF DESCRIPTION OF THE DRAWINGS

These and other aspects, features and advantages of which examples of the invention are capable of will be apparent and elucidated from the following description of examples of the present invention, reference being made to the accompanying drawings, in which:

FIG. 1 is a perspective view of a tissue resection system including a housing, in accordance with at least one example.

FIG. 2 is a perspective view of a cutting window of a cutting assembly in an open state, in accordance with at least one example.

FIG. 3 is a perspective view of a cutting window of a cutting assembly in a closed state, in accordance with at least one example.

FIG. 4 is a diagram of various electronic components of a tissue resection system, in accordance with at least one example.

FIG. 5 is a perspective view of a tissue resection system with a second side of a housing removed, in accordance with at least one example.

FIG. 6 is a perspective view of a tissue resection system with a control board and first rotatable connector removed from a housing, in accordance with at least one example.

FIG. 7 is a perspective view of a drive assembly, in accordance with at least one example.

FIG. 8 is a perspective view of a second rotatable connector and a drive assembly removed from a housing, in accordance with at least one example.

FIG. 9 is a side view of a cutting assembly including an outer cutting member, in accordance with at least one example.

FIG. 10 is a side view of the cutting assembly of FIG. 9 with a distal component of the outer cutting member removed from a proximal component thereof, in accordance with at least one example.

FIG. 11 is a side view of the proximal component and the distal component of FIGS. 9-10, in accordance with at least one example.

FIG. 12 is a side view of a cutting assembly including an outer cutting tube with a cutting edge, in accordance with at least one example.

FIG. 13 is a side view of the outer cutting tube FIG. 12, in accordance with at least one example.

FIG. 14 is a side view of a cutting assembly including an inner cutting tube with an inverse cutting surface, in accordance with at least one example.

FIG. 15 is a side view of the inner cutting tube of FIG. 14, in accordance with at least one example.

### DETAILED DESCRIPTION

Specific examples of the invention will now be described with reference to the accompanying drawings. This invention may, however, be embodied in many different forms and should not be construed as limited to the examples set forth herein; rather, these examples are provided so that this disclosure will be thorough and complete, and will fully convey the scope of the invention to those skilled in the art. The terminology used in the detailed description of the examples illustrated in the accompanying drawings is not intended to be limiting of the invention. In the drawings, like numbers refer to like elements.

Disclosed herein systems and techniques for uterine tissue resection. More specifically, the tissue resection system disclosed herein may include a dual-tube type cutting instrument capable of removing and irrigating tissues from the uterus. Dual-tube type cutting instruments may include a cutting assembly having a stationary outer cutting tube defining an outer cutting window near a distal end, and a driven inner cutting tube received within the outer cutting tube and defining an inner cutting window. In use, such a cutting assembly may be passed through a cylindrical channel of various commercially available hysteroscopes capable of providing irrigation fluid to surgical sites.

In various examples, the tissue resection system disclosed herein can help to address the shortcomings outlined above, among others, by including a swivel interface for a suction source and an internal control board for regulating power.

First, for example, the swivel interface may enable free rotation of a handle of the tissue resection system relative to an external suction source, and more particularly, to a suction tube used to connect the external suction source to the tissue resection system. This may enable an operating physician to move the handle into any rotational position (e.g., between 0 and 360 degrees) with respect to the suction source during alignment of the cutting window of the cutting assembly with target tissue, which may, in turn, help to reduce both the duration and difficulty of a surgical procedure.

Second, the inclusion of a battery, motor, and a control board within a housing of the tissue resection system may eliminate the need for an external power source and/or controller, which may reduce capital cost of the tissue resection system and increase its mobility. Moreover, the control board may be electrically connected to each of the electronic components of the tissue resection devices, including an onboard power source and motor, and may thus integrate various power management features to maximize cutting performance by ensuring the motor consistently receives an optimal operating voltage, and improve both the short-term (e.g., during a single surgical procedure) and long-term life of the power source and the motor.

Third, in some examples, the tissue resection device may also include a cutting assembly having an outer cutting member that receives a rotating and reciprocating inner cutting tube and is comprised of two separate components. More specifically, the outer cutting member may include a proximal component forming a generally tubular shape and a separately formed, and subsequently attached, distal end or tip of the outer cutting member to provide the ability to customize or optimize the geometry of the outer cutting tube in the region or area where tissue cutting action occurs, to thereby improve cutting performance and/or efficiency, and concurrently preserve the ability to manufacture the remaining portion of the outer cutting member using economical techniques or methods to limit any increase in manufacturing cost.

Fourth, in some examples, the tissue resection device may further include a cutting assembly having a rotating and reciprocating inner cutting tube that is receivable within an outer cutting tube and that includes an inverse cutting surface tapering radially outwardly from an inner surface to an outer surface thereof, to thereby provide a cutting edge having a similar diameter to the outer surface of the inner cutting tube. This may significantly reduce the clearance of the cutting edge within the outer cutting tube over typical or traditional inwardly tapered or chamfered cutting tubes, and as such, may also provide improved cutting performance and/or efficiency without significantly increasing manufacturing cost.

In view of the above, the uterine tissue resection system disclosed herein may provide benefits to both patients and physicians.

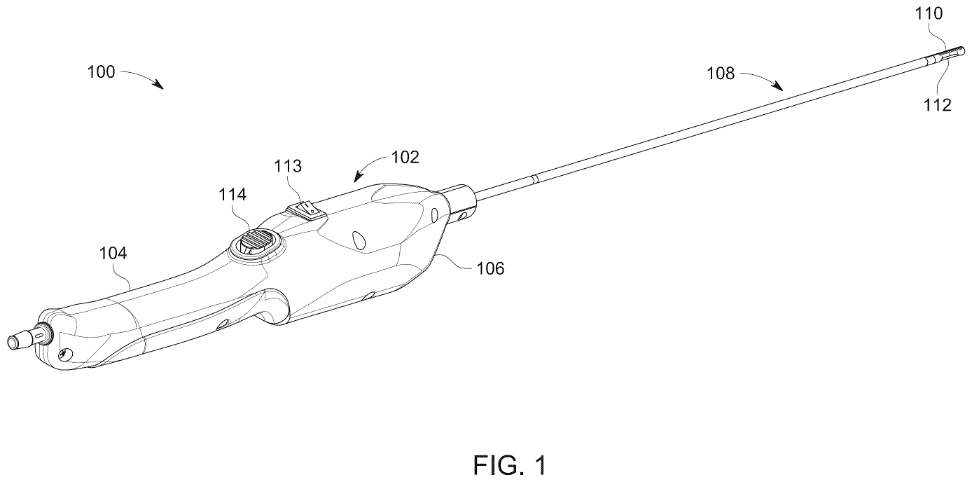

FIG. 1 is a perspective view of a tissue resection system 100 including a housing 102 sized and shaped to function as a handle for a user. As shown in FIG. 1, the housing 102 may include a first side 104 and a second side 106. Extending from the housing 102 may be a cutting assembly 108, which may include an outer cutting tube 110 with an inner cutting tube 112 received therein. The outer cutting tube 110 may be fixedly connected (i.e., affixed) to the housing 102, and may define a closed distal end 111, a first lumen 115, and an outer cutting window 117 positioned proximally thereto.

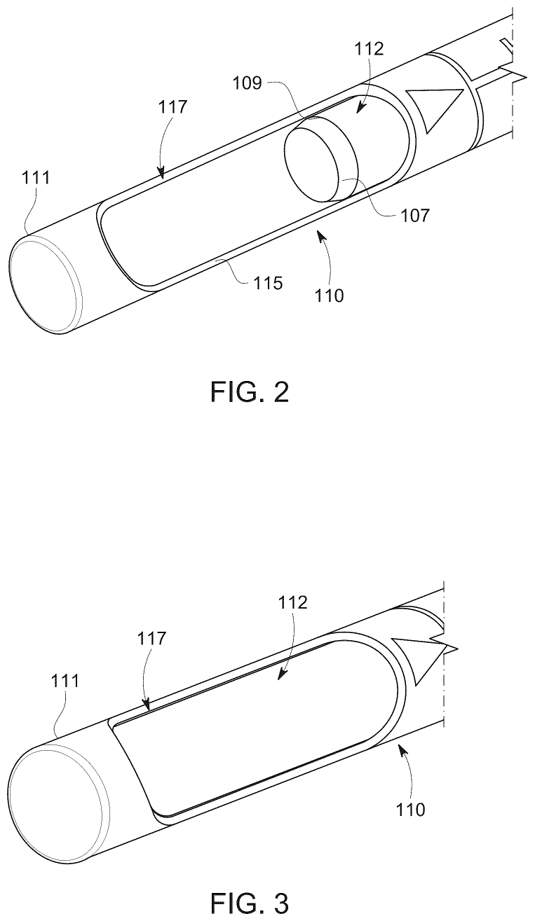

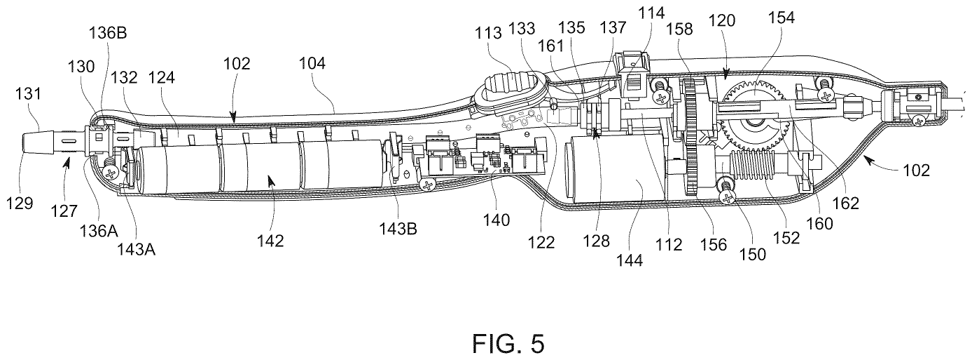

The inner cutting tube 112 may, by virtue of being connected to a drive assembly 120 (FIGS. 5-8), rotate within the outer cutting tube 110 while simultaneously or concurrently reciprocating between an extended position and a retracted position. For example, FIG. 2 illustrates the inner cutting tube 112 in a partially retracted position within the outer cutting tube 110 to reveal a distal end 109 and a second lumen 107 of the inner cutting tube 112, and FIG. 3 illustrates the inner cutting tube 112 in a fully extended position within the outer cutting tube 110. In the extended position, the outer cutting window 117 may be completely blocked by the inner cutting tube 112, and in the retracted position, the distal end 109 of the inner cutting tube 112 may be located proximal to the outer cutting window 117 of the outer cutting tube 110.

In some examples, the cutting assembly 108 and the drive assembly 120 may be, or may be similar to, the examples of a worm gear drive shown and/or described in U.S. Patent Publication No. 2020/0268404, filed Feb. 24, 2020, and entitled Apparatus and Method for Removal of Intrauterine Fibroid Formations, which is hereby incorporated by reference in its entirety. In other examples, as discussed further below, the drive assembly 120 may differ from the drive assembly of that publication in one or more aspects.

The tissue resection system 100 may also be adapted for use with the working channels of a hysteroscope, such as, but not limited to, the examples of a hysteroscope shown and/or described in U.S. Patent Publication No. 2019/0133640, filed Nov. 9, 2018, and entitled Rotary Instruments and Methods for Intrauterine Tissue Resection, which is hereby incorporated by reference in its entirety.

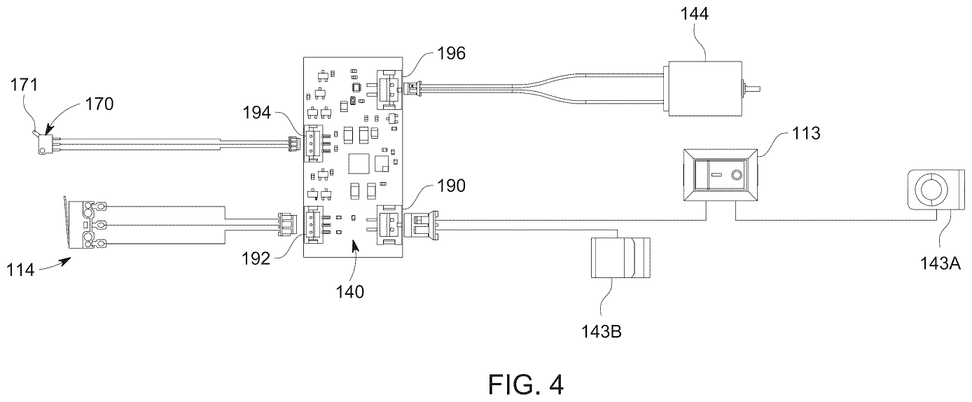

The tissue resection system 100 may include a power switch 113 and an activation switch 114. The power switch 113 and the activation switch 114 may each be positioned to be within reach for a user when grasping the housing 102. The power switch 113 may comprise an on/off toggle switch electrically connected to a power source 142 (FIG. 5) located within the housing 102. When in a closed position, the power switch 113 may enable various electrical components of the tissue resection system 100 to receive power from a power source (FIGS. 4-5). The activation switch 114 may be a momentary on/off switch that must be depressed or otherwise continuously engaged by a user, to maintain the activation switch 114 in a closed position.

With the activation switch 114 in a closed position, a motor 144 (FIGS. 4-5) may be active. Thus, the power switch 113 may function to prevent activation of the tissue resection system 100 in the event the activation switch 114 is accidentally depressed. The tissue resection system 100 may also include a safety switch 170. The safety switch 170 may be adapted, as described in detail further below, to ensure that the inner cutting tube 112 stops in its fully extended position each time the activation switch 114 is released.

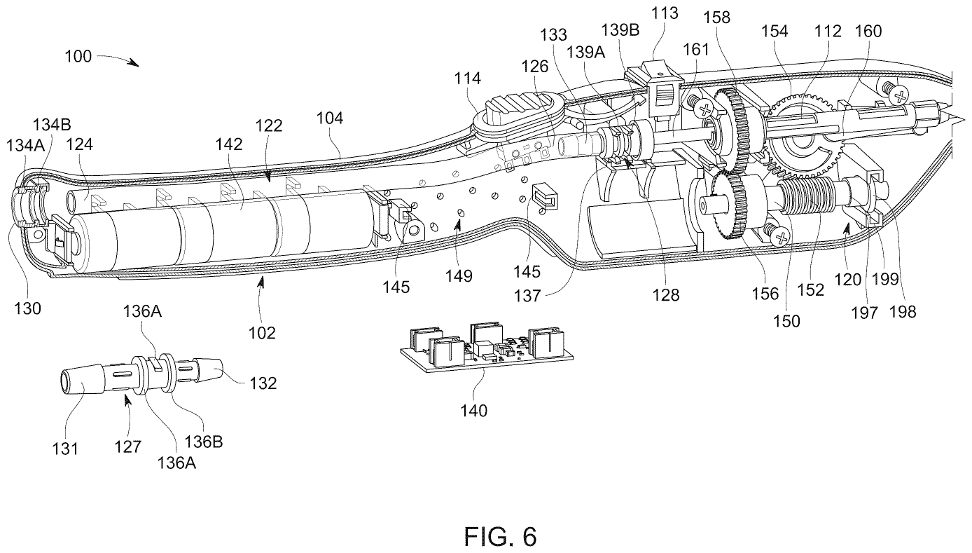

To enable the above and other functions, the tissue resection system 100 may include a control board 140 such as shown in FIGS. 4-5. The control board 140 may, in various examples, comprise a printed circuit board including a microcontroller or microprocessor. The control board 140 may be maintained in place within the housing 102 via a plurality of mounting bosses 145 (FIG. 6). In some such examples, the housing 102 may further include a plurality of apertures 149 (FIG. 6) configured to help reduce or dissipate heat generated by the control board 140, the power source 142, and/or the motor 144.

The control board 140 may be electrically connected to all the electronic components of the tissue resection system 100 located within the housing 102. For example, the power switch 113, the activation switch 114, and the safety switch 170 may be connected to the control board 140 through a first port 190, a second port 192, and a third port 194, respectively, such as shown in FIG. 4.

Further, the motor 144 (FIGS. 4-5) may be electrically connected to the control board 140 through a fourth port 196 (FIG. 4), and the power source 142 (FIGS. 4-6) may be electrically connected to the control board 140 through both the first port 190 and the power switch 113. In one such example, a negative terminal 143B (FIGS. 4-5) of the power source 142 may be connected to the control board 140 through the first port 190, and a positive terminal 143A (FIGS. 4-5) may be connected to the control board 140 through the power switch 113.

In some examples, the power source 142 may comprise one or more individual batteries, such as, but not limited to, one, two, three, four, five, six, or more individual batteries. In one example, such as shown in FIG. 4, the power source 142 may comprise three individual cells. In one example, the power source 142 may be a single 9V cell. In another example, such as shown in FIG. 4, the power source 142 may be comprised of three 3V cells. While several examples are disclosed above, one skilled in the art will realize that other types of batteries may be used. The motor 144 may generally be an electric motor adapted to create high-speed rotary motion. In some examples, the motor 144 may be adapted to rotate at between, but not limited to, about 4,000 and about 12,000 revolutions per minute.

The control board 140 may be configured to integrate various power management features to ensure efficient, safe, and reliable operation of motor 144 and the power source 142. For example, the control board 140 may monitor the voltage, temperature, and/or state of charge of the battery cell(s) of the power source 142 to prevent overcharging or over-discharging and may also ensure that the battery cell(s) of the power source 142 are evenly drawn down or discharged during use. This may significantly increase both the life of the power source 142 during a surgical procedure, as well as extend the long-term health of the battery cell(s). The control board 140 may also precisely regulate the delivery of power to the motor 144 to help increase the life of, of the level of control over, the motor 144, as well as maximize cutting performance by ensuring the inner cutting tube 112 always applies a consistent cutting force to tissue. Still further, the control board 140 may be configured to cease power to the power switch 113, the activation switch 114, and the safety switch 170 in the event of a system short to thereby protect each of these switches from damage.

As one of skill in the art will appreciate, the inclusion of the control board 140 into the housing 102 of the tissue resection system 100, may help to increase the longevity of the power source 142 during a surgical procedure while preserving, as compared to devices requiring external power supplies and controllers, a low capital cost and high level of mobility. In some examples, the control board 140 may further enable a lower-power safety cut-off feature to be integrated. For example, control board 140 may be adapted to, upon sensing a state of change of the power source 142 is critically low, stop the inner cutting tube 112 in the extended position (e.g., when the safety switch 170 is open) to ensure the outer cutting window 117 is closed for safe for removal of the cutting assembly 108 from a patient.

Next, as previously noted, the tissue resection system 100 may include a swivel connection interface adapted to enable free rotation of the housing 102 relative to an external suction source. As shown in FIGS. 5-6, such a swivel connection interface may be realized in the form of a first swivel connection 127 and/or a second swivel connection 128. The first swivel connection 127 may be comprised of a rotatable interface formed between a proximal end 124 of a suction tube 122 (drawn in shadow in FIGS. 5-6) and a first connector 129 retained by the housing 102. For example, the proximal end 124 of the suction tube 122 may be engaged by a proximal barb nipple 132 of the first connector 129, and the first connector 129 may be rotatably received within a first mounting boss 130 defined by the housing 102.

The first connector 129 may include a plurality of protrusions 136A and an annular projection 136B, and the first mounting boss 130 may include a first pair of annular recesses 134A and 134B configured to axially retain, and enable free rotation of, the plurality of protrusions 136A and the annular projection 136B. The first connector 129 may further include a distal barb nipple 131 adapted to engage a suction tube (not shown) of a wide variety of pre-existing suction sources or vacuum generators to fluidly couple the tissue resection system 100 thereto. In some examples, the first swivel connection 127 may enable a user to freely rotate the housing 102 at least 180 degrees with respect to the first connector 129 before the suction tube 122 resists further rotation.

The second swivel connection 128 may be comprised of a rotatable interface formed between a proximal end 126 of the suction tube 122 and a second connector 135 retained by the housing 102. For example, the proximal end 126 of the suction tube 122 may be engaged by a distal barb nipple 133 of the second connector 135, and the second connector 135 may be rotatably received within a second mounting boss 137 defined by the housing 102.

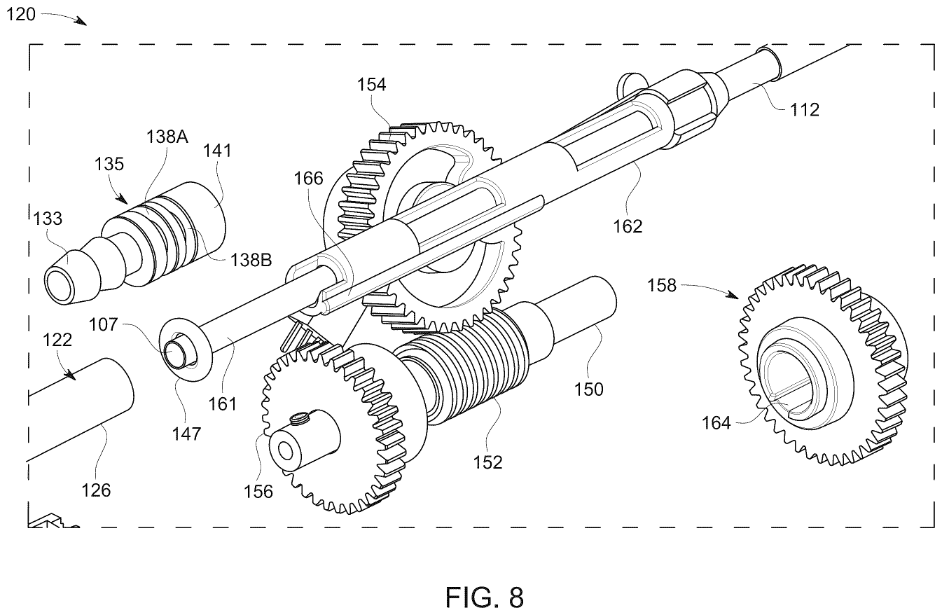

The second connector 135 may further include a second pair of annular recesses 138A and 138B, and the second mounting boss 137 may include a pair of projections 139A and 139B configured to axially retain, and enable free rotation of, second pair of annular recesses 138A and 138B. The second connector 135 may further include a proximal end portion 141 adapted to receive a distal end portion 161 of the inner cutting tube 112 to fluidly couple the inner cutting tube 112 to the suction tube 122, and thereby an external suction or vacuum source. In some examples, the proximal end portion 141 (FIG. 8) of the second connector 135 may further be adapted to receive a seal 147 (FIG. 8) to establish an air or liquid tight barrier between the second connector 135 and the inner cutting tube 112.

In view of the above, the first swivel connection 127 and the second swivel connection 128 may enable an operating physician to grasp, and freely rotate, the housing 102 to align the outer cutting window 117 with target tissue to be removed during a surgical procedure. For example, one skilled in the art will appreciate that as the housing 102 begins to rotate with respect to a suction tube extending to the suction source from the distal barb nipple 131, the first connector 129, and in turn the suction tube 122 and the second connector 135 fixedly connected thereto, may rotate within the housing 102 to prevent torsional forces, and thereby resistance, from accumulating and restricting the rotation of the housing 102.

The drive assembly 120 may, in some examples, include a drive shaft 150, a worm 152, a worm gear 154, a first spur gear 156, a second spur gear 158, a crank 160, and a sleeve 162.

As shown in FIGS. 5-8, the drive shaft 150 may be connected to the motor 144 and carry the first spur gear 156 and the worm 152. In some examples, a distal end 197 of the drive shaft 150 may be received within, and supported by, a bushing 198 retained by a retaining boss 199 defined by the housing 102. In this regard, the bushing 198 may be adapted to freely rotate within the retaining boss 199 during rotation of the drive shaft 150. In some examples, the bushing 198 may be made from metallic materials, such as, but not limited to, aluminum, steel, stainless steel, or titanium, among various metallic alloys. In other examples, the bushing 198 may be made from softer material, such as a polymeric or elastomeric material including, but not limited to, acrylonitrile butadiene styrene (ABS), nylon or polyamide (PA), polyoxymethylene, or rubber, among others. As may be appreciated, the use of such relatively soft materials for the bushing 198 may help to dampen vibration and reduce noise during operation of the drive assembly 120 caused by activation of the motor 144.

The worm gear 154 may mesh with the threading of the worm 152, and the second spur gear 158 may mesh with the teeth of the first spur gear 156. The crank 160 may be attached to both the worm gear 154, at a non-centric location, and to the inner cutting tube 112. The inner cutting tube 112 may be connected to the second spur gear 158 through the sleeve 162. More specifically, the second spur gear 158 may define a longitudinal groove 164 and the sleeve 162 may define a longitudinal projection 166. The longitudinal groove 164 may be configured to receive the longitudinal projection 166 to enable the sleeve 162 to slide proximally and distally within the second spur gear 158 while concurrently receiving rotational drive from the second spur gear 158.

In the operation of some examples, activation of the motor 144, such as resulting from the power switch 113 and the activation switch 114 being closed and the control board 140 allowing power to flow to the motor 144 from the power source 142, may cause the drive shaft 150, the first spur gear 156, and the worm 152 to begin rotating. The rotation of the worm 152 may then turn the worm gear 154; which may cause the sleeve 162 and the inner cutting tube 112 attached thereto to axially reciprocate between an extended position and a retracted position as a result of non-centric movement of the crank 160 about the worm gear 154. Simultaneously, the rotation of the first spur gear 156 may cause the second spur gear 158 to rotate, resulting in rotation of the inner cutting tube 112 via engagement of the sleeve 162 with the second spur gear 158. Thus, concurrent reciprocation and rotation of the inner cutting tube 112 may be achieved.

In this regard, it may also be appreciated that the drive ratio of the worm 152 may be selected based upon an intended use of the tissue resection system 100. For example, in an example polypectomy procedure involving the removal of soft, gelatinous tissues, the worm 152 may be configured at a lesser or lower gear reduction ratio as the need for gear reduction and torque multiplication is lesser when cutting through relatively soft tissues. In one such example, the worm 152 may be configured such that it forms a gear ratio of 42:2 with the motor 144. In other words, for every 21 revolutions of the motor 144, the inner cutting tube 112 will complete one reciprocation cycle within the outer cutting tube 110. In such an example, such as at an example motor speed of about 7400 revolutions per minute (“RPM”), the inner cutting tube 112 will fully reciprocate or cycle around 350, or more specifically, 342.4, times per minute. However, in other examples, it is appreciated that a wide variety of other gear ratios between the worm 152 and the motor 144 may be selected based upon the type of tissue to be removed, as well as a wide variety of other design factors.

In another example, such as in a myomectomy procedure involving the removal of harder, denser tissue, the worm 152 may be configured at a greater or higher gear reduction ratio, as the need for gear reduction and torque multiplication is greater when cutting through relatively hard or dense tissues. In one such example, the worm 152 may be configured such that it forms a gear ratio of 42:1 with the motor 144. In such an example, for every 42 revolutions of the motor 144, the inner cutting tube 112 will complete one reciprocation cycle within the outer cutting tube 110. Additionally, in such an example, such as at an example motor speed of about 7400 revolutions per minute (“RPM”), the inner cutting tube 112 will fully reciprocate or cycle about 176 times per minute. However, in other examples, it is appreciated that a wide variety of other gear ratios between the worm 152 and the motor 144 may be selected based upon the type of tissue to be removed, as well as a wide variety of other design factors.

In some examples, such a doubling or a halving of a gear reduction ratio between the worm 152 and the motor 144, such as from doubling the total gear reduction from 42:2 to 42:1, may be accomplished by replacing a worm 152 defining a double-thread with a replacement worm 152 which defines a single-thread. In this regard, the worm 152 may be easily and conveniently replaced by a user, or selected during manufacturing of the tissue resection system 100, to thereby selectively configure the tissue resection system 100 for resecting relatively soft tissues or resecting relatively hard tissues.

In contrast to the third switch of the drive assembly 120, the safety switch 170 of the tissue resection system 100 may be configured to engage the worm gear 154. For example, the worm gear 154 may include a surface 168 defining geometry adapted to move a lever 171 of the safety switch 170 to thereby open and close the safety switch 170 during rotation of the worm gear 154. In some examples, the surface 168 may comprise a semi-circular segment 172 (FIG. 6) and an inset segment 174 (FIG. 6). In one such example, the semi-circular segment 172 may extend between about 250 degrees and about 290 degrees around an axis of rotation of the worm gear 154. In one example, the semi-circular segment 172 may extend about 270 degrees around an axis of rotation of the worm gear 154.

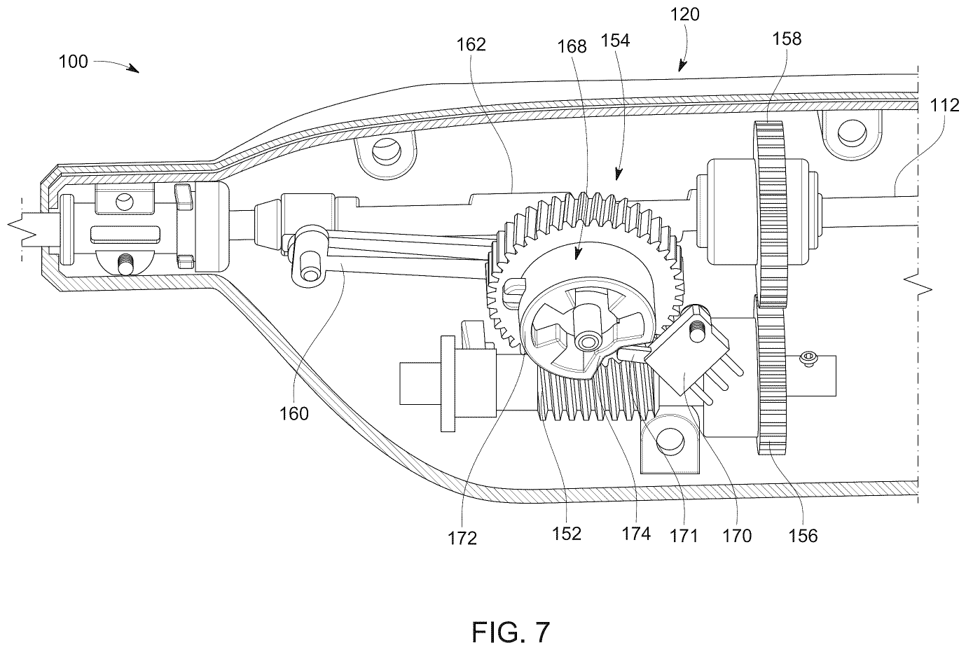

The inset segment 174 may be an area of the surface 168 that is radially inset, such toward a center of the worm gear 154, from the semi-circular segment 172. The inset segment 174 may be sized and shaped to cause the lever 171 to move from a closed position to an open position when the lever 171 enters the inset segment 174, and subsequently return to a closed position upon returning to engagement with the semi-circular segment 172. As one of skill in the art will appreciate, the crank 160 may be attached to the worm gear 154 in an angular position selected to ensure the lever 171 is in contact with the inset segment 174 when the inner cutting tube 112 is in its extended position, such as shown in FIG. 7.

Such a configuration may allow the safety switch 170 to ensure the inner cutting tube 112 always reassumes the fully extended position when the activation switch 114 is released. For example, because the safety switch 170 and the activation switch 114 may be connected to the motor 144 through the control board 140, power may be selectively supplied to the motor 144 until each of the activation switch 114 and the safety switch 170 are open. This may only occur when the activation switch 114 is released by a user, and the inner cutting tube 112 reaches a fully extended position causing the lever 171 to open the safety switch 170 as it engages the inset segment 174.

Thus, when the motor 144 is not in an active state, and the inner cutting tube 112 blocks the outer cutting window 117 to ensure that no tissue can be inadvertently damaged by being drawn into the cutting window as a result of suction remaining in the suction tube 122 or inner cutting tube 112. Moreover, because the inner cutting tube 112 blocks the outer cutting window 117 when the motor 144 is not in an active state, the total amount of irrigation fluid consumed during a procedure can be minimized. For example, since only a relatively small volume of previously introduced irrigation fluid is suctioned out of a patient through the outer cutting window 117 when the outer cutting window 117 is closed, the total volume of irrigation fluid introduced into a patient through a hysteroscope used in conjunction with the tissue resection system 100 may be significantly reduced.

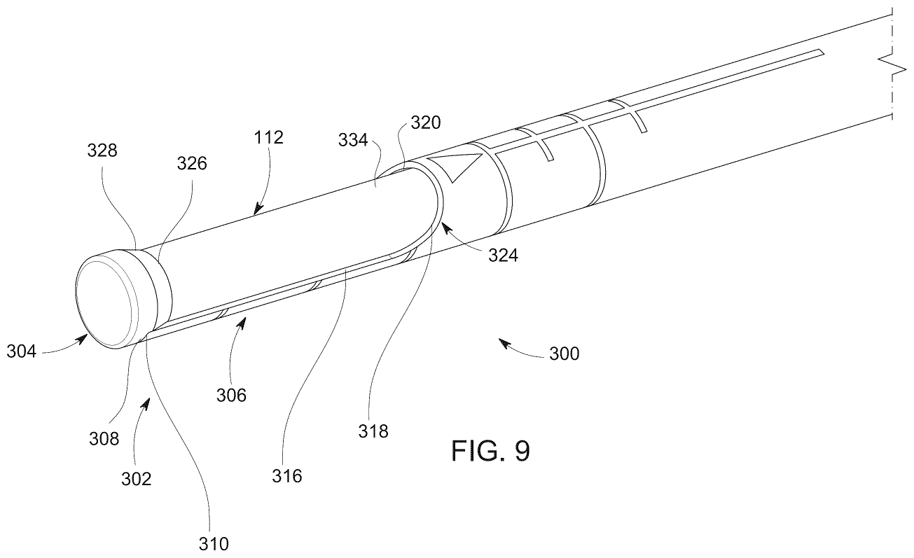

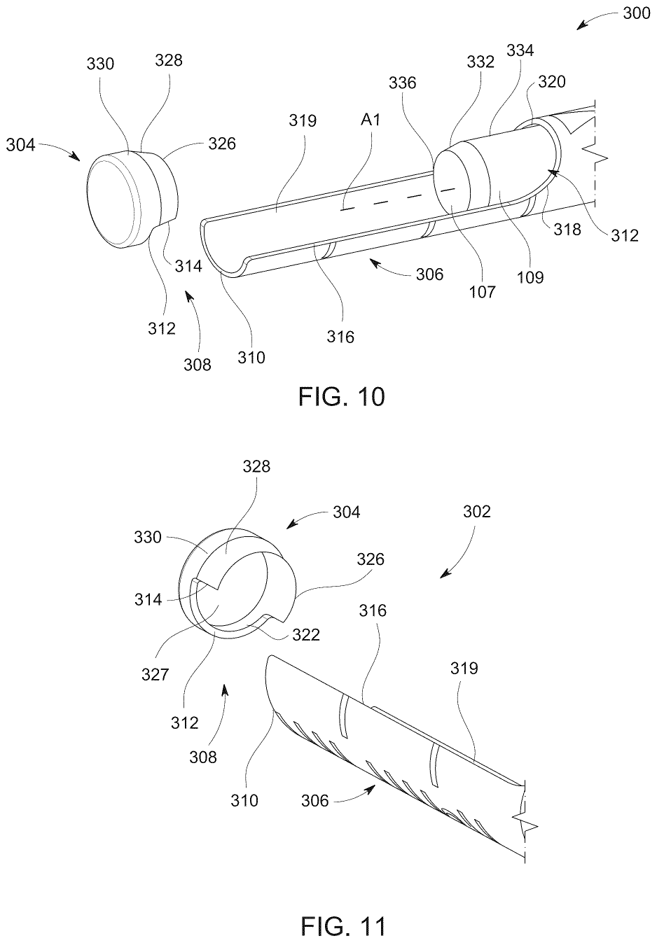

Additionally, as previously noted above and as shown in FIGS. 9-11, the tissue resection system 100 may include a cutting assembly 300 including an outer cutting member 302 comprised of a distal component 304 and a proximal component 306, and the inner cutting tube 112. The outer cutting member 302 may be similar to the outer cutting tube 110 previously described above, except in that the outer cutting member 302 may be collectively formed by the distal component 304 and the proximal component 306.

In this regard, the distal component 304 may be manufactured separately from the proximal component 306 and then subsequently affixed thereto. In various examples, the distal component 304 and the proximal component 306 may each be manufactured using similar, or different, construction techniques, such as including, but not limited to, subtractive manufacturing (e.g., machining such as milling, turning, and/or drilling, electrical discharge machining, laser cutting, or waterjet cutting), additive manufacturing (e.g., three-dimensional printing), casting, molding, or joining and fabrication (e.g., forging, rolling, hydroforming, extrusion, or stamping).

However, as the proximal component 306 forms a largely tubular and comparatively simplistic shape, as compared to the distal component 304, the proximal component 306 may be manufactured using less expensive and/or less complex construction techniques which may typically or traditionally be used to economically produce tubular structures. In one specific example, the distal component 304 may be manufactured using conventional machining, electrical discharge machining, laser ablation, and/or or a combination of the aforementioned techniques, and the proximal component 306 may be manufactured primarily using at least extrusion fabrication and/or may be finished with minimal additional machining or modification, such as to include the cutout 318 described further below.

The distal component 304 may then be attached, affixed, or otherwise secured to the proximal component 306 using various attachment methods, such as, but not limited to, any of welding (e.g., MIG welding, TIG welding, spot or resistance welding, laser welding, or friction welding), adhesives and/or chemical bonds, interference or press-fitting, or via a threaded interface formed therebetween. The distal component 304 and the proximal component 306 may also be made from various metallic materials, such as, but not limited to, aluminum, steel, stainless steel, or titanium, among various metallic alloys.

In this regard, the distal component 304 may include an engaging surface 308 sized and shaped to contact, and conform to for attachment purposes, to a distal end surface 310 of the proximal component 306. In some examples, the engaging surface 308 may only include only a first portion 312. The first portion 312 may be adapted to conform to a distal end surface 310 of the proximal component 306. For example, the first portion 312 may form a corresponding, or a similarly sized and shaped, cross-sectional shape to the distal end surface 310, such that the first portion 312 may be in continuous surface contact with the distal end surface 310 when the distal component 304 is affixed to the proximal component 306.

In some specific examples, the first portion 312 and the distal end surface 310 may each define semi-circular cross-sectional shapes that extend about 180 degrees around a central axis A1 extending concentrically through the inner cutting tube 112 and the outer cutting member 302. In other examples, the first portion 312 and the distal end surface 310 may define other cross-sectional shapes that may extend within an inclusive range of about 45 degrees and about 270 degrees around the central axis A1.

Further, the first portion 312 and the distal end surface 310 may each extend, or may otherwise be orientated at, similar or different angles relative to the central axis A1. For example, the first portion 312 and the distal end surface 310 may extend orthogonally to the central axis A1. However, in other examples, the first portion 312 and the distal end surface 310 may form various acute or obtuse angles relative to a single point or tangent along the central axis A1, such as, but not limited to, angles within an inclusive range of about 1 degree to about 179 degrees. In such examples, it is appreciated that the collective sum of the angle that the first portion 312 forms relative to the central axis A1 and the angle that the distal end surface 310 forms relative to the central axis A1 will equal 180 degrees such that the first portion 312 and the distal end surface 310 may be in continuous surface contact with each other.

In some examples, the engaging surface 308 may also include second portion 314. The second portion 314 may be adapted to help locate and position the distal component 304 on the proximal component 306, as well as help provide additional surface engagement and/or bonding area between the distal component 304 and the proximal component 306. For example, the second portion 314 may be sized and shaped to be in continuous surface contact with an upper surface 316 of the proximal component 306 defined by a cutout 318 in the proximal component 306. The cutout 318 may expose an inner surface 319 of the proximal component 306 and may extend proximally from the distal end surface 310 to a first lumen 320 adapted to receive the inner cutting tube 112 and defined by the inner surface 319. Additionally, as is apparent from FIGS. 9-11, the second portion 314 may generally form, or be comprised of, two parallel surface areas which extend substantially parallel to the central axis A1 and that extend proximally along the upper surface 316 of the proximal component 306 when the distal component 304 is placed on the proximal component 306.

As may be appreciated in view of all the above, once the engaging surface 308 is positioned on the distal end surface 310 and/or the upper surface 316 of the proximal component 306, the distal component 304 may be affixed to the proximal component 306, such as via one or more laser welds which trace or otherwise follow an interface (i.e. a boundary or region where the two surfaces meet) between the engaging surface 308 and the proximal component 306, and/or an adhesive or chemical bond formed between the engaging surface 308 and the proximal component 306.

The distal component 304 also includes an inner surface 322. The inner surface 322 may be adapted to extend 360 degrees around the central axis A1. Additionally, the inner surface 322 may be oriented concentrically with the inner surface 319, and the first lumen 320 defined thereby, or the central axis A1. In this respect, the inner surface 322 may be sized and shaped to enable the distal component 304 to receive the distal end 109 of the inner cutting tube 112 therein. For example, when the distal component 304 is affixed to the proximal component 306 and the inner cutting tube 112 is in a fully extended or distal-most position within the outer cutting member 302, the inner surface 322 may circumferentially encompass the distal end 109 of the inner cutting tube 112.

Thus, when the inner cutting tube 112 rotates and axially reciprocates within the outer cutting member 302, as previously explained above, the distal end 109 of the inner cutting tube 112 may, when moving in a distal direction, exit the first lumen 320, traverse the cutout 318, and enter an area of the distal component 304 defined by the inner surface 322 and an end surface 327 thereof. Therefore, it may be readily appreciated that when the distal component 304 is affixed to the proximal component 306, the cutout 318 and the distal component 304 collectively form an outer window 324 that is opened and closed by axial translation of the inner cutting tube 112 during operation of the tissue resection system 100.

In some examples, the inner surface 322 may be adapted to enable the cutting assembly 300 to possess improved performance characteristics. For example, the inner surface 322 may define a maximum diameter or circumference that is smaller than a maximum diameter or circumference defined by the inner surface 319 of the proximal component 306. Therefore, the distal component 304 may possess a smaller radial gap between the outer surface 334 and/or the distal end 109 of the inner cutting tube 112 and the inner surface 322, to thereby help reduce radial clearance at the location(s) along the cutting assembly 300 where tissue cutting or shearing action primarily occurs (i.e. at or near the first cutting edge 326 and/or the second cutting edge 336 discussed further below), and a significantly a larger radial gap between the inner surface 319 and/or the first lumen 320 of the proximal component 306 and the outer surface 334 and/or the distal end 109 of the inner cutting tube 112 (i.e. the outer surface 334), to thereby increase radial clearance at the location(s) along the cutting assembly 300 where tissue cutting action does primarily not occur, to in turn improve performance characteristics of the cutting assembly 300.

In some such examples, a radial gap or distance defined between the outer surface 334 of the inner cutting tube 112 and the inner surface 322 of the distal component 304 may measure within, but not limited to, an inclusive range of about 0.001 inches and about 0.003 inches. In some examples, a radial gap or distance defined between the outer surface 334 of the inner cutting tube 112 and the inner surface 319 of the proximal component 306 may measure within, but not limited to, an inclusive range of about 0.003 inches and about 0.006 inches. In some examples, a radial gap or distance defined between the outer surface 334 and the inner surface 322 may measure between 0.0005 to 0.003 inches, and a radial gap or distance defined between the outer surface 334 and the inner surface 319 may measure within an inclusive range of about 0.003 inches and about 0.006 inches.

In some specific examples, a difference between a radial gap or distance defined between the outer surface 334 and the inner surface 322, and a radial gap or distance defined between the outer surface 334 and the inner surface 319, may measure within an inclusive range of about 0.001 inches to about 0.005 inches. In one such specific example, a radial gap or distance defined between the outer surface 334 and the inner surface 322 may measure about 0.002 inches, and a radial gap or distance defined between the outer surface 334 and the inner surface 319 may measure about 0.004 inches, such that the difference between the radial gap or distance defined between the outer surface 334 and the inner surface 322, and the radial gap or distance defined between the outer surface 334 and the inner surface 319, measures about 0.002 inches.

Moreover, with respect to geometry adapted to enable the cutting assembly 300 to possess improved performance characteristics, the distal component 304 may also define a first cutting edge 326 that is sharpened to cut or otherwise sever tissue. The first cutting edge 326 may be proximal-most end surface of the distal component 304. In this respect, the first cutting edge 326 may be defined at a meeting point or interface between a first sloped surface 328 which tapers inwardly, in a proximal direction, from an outer surface 330 of the distal component 304 to the inner surface 322 of the distal component 304. The first sloped surface 328 may form various acute angles, in a proximal direction, relative to a single point or tangent along the central axis A1. In some examples, the first sloped surface 328 may form an angle within an inclusive range of about 5 degrees and about 50 degrees. In one specific example, the first sloped surface 328 may form an angle of about 15 degrees relative to the central axis A1. In any such examples, it is further appreciated that the sharpness of the first cutting edge 326 may be increased by reducing the angle that the first sloped surface 328 forms relative to a single point or tangent along the central axis A1.

Additionally, as is apparent from FIGS. 9-11, the first cutting edge 326 may terminate laterally at the second portion 314 of the engaging surface 308. In this regard, the collective sum of an angular distance (in degrees) which the distal end surface 310 extends about the central axis A1 and the angular distance which the first cutting edge 326 and the first sloped surface 328 extends about the central axis A1 may equal 360 degrees. For example, if the distal end surface 310 of the proximal component 306 extends around the central axis A1 by about 180 degrees, the first cutting edge 326 will also extend around the central axis A1 by about 180 degrees. However, in other examples, it is appreciated that the first cutting edge 326 and/or the first sloped surface 328 may extend within an inclusive range of about 45 degrees and about 270 degrees around the central axis A1, depending upon the angular distance which the distal end surface 310 extends about the central axis A1.

Similarly, as previously noted, the distal end 109 of the inner cutting tube 112 may be adapted to cut or otherwise sever tissue. More specifically, the distal end 109 may define a second sloped surface 332 which tapers inwardly, in a distal direction, from an outer surface 334 to the second lumen 107 to define a second cutting edge 336. The second sloped surface 332 may form various acute angles, in a distal direction, relative to a single point or tangent along the central axis A1. In some examples, the first sloped surface 328 may form angle within an inclusive range of about 5 degrees and about 50 degrees. In one specific example, the second sloped surface 332 may form an angle of about 15 degrees relative to the central axis A1. In any such examples, it is further appreciated that the sharpness of the second cutting edge 336 may be increased by reducing the angle that the second cutting edge 336 forms relative to a single point or tangent along the central axis A1.

In some examples, the first sloped surface 328 and the second sloped surface 332 may form similar angles relative to one another. In one such example, the first sloped surface 328 and the second sloped surface 332 may each form an angle of about 15 degrees relative to the central axis A1. However, in other examples, the first sloped surface 328 and the second sloped surface 332 may form different angles relative to one another. In some examples, the surface area of one or more of the first cutting edge 326, the second cutting edge 336, the first sloped surface 328, or the second sloped surface 332, may be smooth or polished, or alternatively, the surface area of the first cutting edge 326, the second cutting edge 336, the first sloped surface 328, or the second sloped surface 332 may be serrated, such as by including a variety of different cutting projections or protrusions.

In view of all the above, the outer cutting member 302 may provide several benefits and/or advantages for the tissue resection system 100, among other tissue resection devices. More specifically, the distal component 304 and the proximal component 306, by virtue of being separately formed components, may enable the distal component 304 to possess improved surface features and/or geometry than may otherwise be feasibly implemented into a unitary outer cutting member, while also retaining the ability to utilize traditional and/or more economical construction techniques for the majority of the outer cutting member 302 (e.g., the proximal component 306) to thereby minimize any increase in manufacturing cost.

Moreover, first, with respect to such surfaces features and/or geometry, the inner surface 322 may enable the distal component 304 to provide a tighter fit with the distal end 109 and/or the outer surface 334 of the inner cutting tube 112 and the inner surface 319 may enable the proximal component 306 to provide a looser fit with the distal end 109 and/or the outer surface 334 of the inner cutting tube 112. In this regard, because the radial clearance or gap between the inner cutting tube 112 and the outer cutting member 302 is relatively tight only where tissue cutting or shearing action occurs (e.g., at or near the first cutting edge 326 and/or the second cutting edge 336), increased cutting efficiency and consistency may be achieved without substantially increasing friction between the inner cutting tube 112 and the outer cutting member 302. Second, with respect to such surfaces features and/or geometry, as both a distal end (i.e. the first cutting edge 326 of the distal component 304) of the outer window 324, and a distal end (i.e. the second cutting edge 336) of the inner cutting tube 112, may be sharpened to cut or sever tissue, cut quality and precision, as well as cutting efficiency, may be further enhanced.

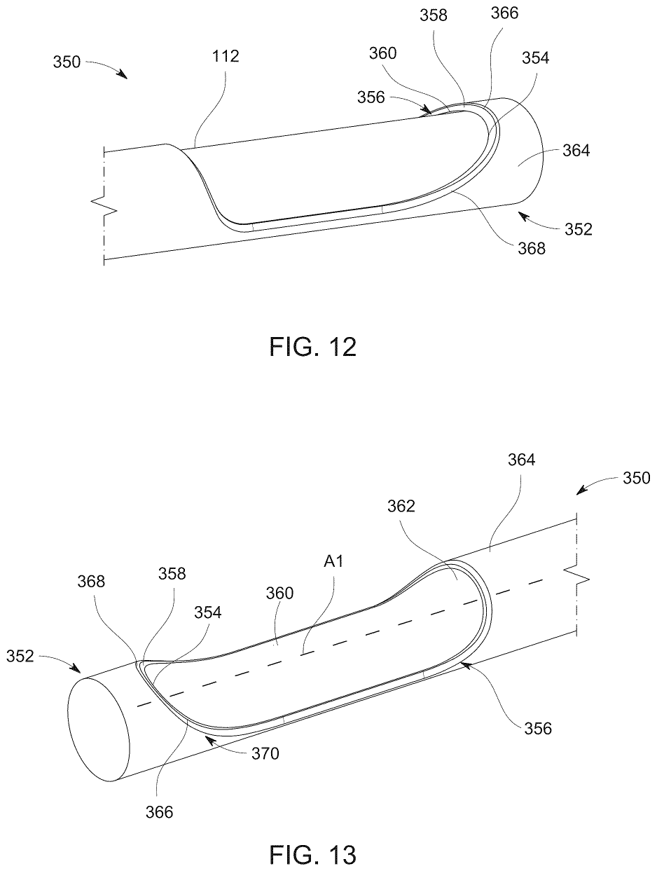

Next, as shown in FIGS. 12-13, in some examples the tissue resection system 100 may include a cutting assembly 350. The cutting assembly 350 may include the inner cutting tube 112, or the inner cutting tube 402 described further below, and an outer cutting tube 352 including a cutting edge 354. The outer cutting tube 352 may be similar to the outer cutting tube 110 previously discussed above, except in that the outer cutting tube 352 may include an outer window 356 at least partially defined by the cutting edge 354.

The cutting edge 354 may be a sharpened surface adapted for cutting or severing tissue. The cutting edge 354 may, in some examples, be defined at a sharpened meeting point or interface between a first sloped surface 358 which tapers inwardly, in a proximal direction, and an inner surface 360 defining a first lumen 362 of the outer cutting tube 352. In some examples, the first sloped surface 358 may extend from the cutting edge 354 to an outer surface 364 of the outer cutting tube 352. However, in other examples, the first sloped surface 358 may extend to a ridge 366 defined at a meeting point or interface between the first sloped surface 358 and a second sloped surface 368 of the outer cutting tube 352.

The first sloped surface 358 may form various acute angles, in a proximal and/or distal direction, relative to a single point or tangent along a central axis A1 extending concentrically through the inner cutting tube 112 and the outer cutting tube 352. In some examples, the first sloped surface 358 may form an angle within an inclusive range of about 5 degrees and about 50 degrees relative to the central axis A1. In one specific example, the first sloped surface 358 may form an angle of about 15 degrees relative to the central axis A1. In this regard, it is further appreciated that the sharpness of the cutting edge 354 may be increased by reducing the angle that the first sloped surface 38 forms relative to a single point or tangent along the central axis A1. In some examples, the surface area of the cutting edge 354 and/or the first sloped surface 358 may be smooth or polished, or alternatively, the surface area of surface area of the cutting edge 354 and/or the first sloped surface may be serrated, such as by including a variety of different cutting projections and/or protrusions.

In some examples, the cutting edge 354 may circumferentially surround and/or entirely define the outer window 356. In other examples, the cutting edge 354 may extend around only a portion of the outer window 356, such as, but not limited to, a distal portion 370 of the outer window 356 where cutting action between the inner cutting tube 112 and the outer cutting tube 352 primarily occurs. In such examples, it may be appreciated that any remaining area of the interface, intersection, or a meeting point between the first sloped surface 358 and the inner surface 360 that is not sharpened to define the cutting edge 354 may instead define a relatively blunt edge.

The outer cutting tube 352 may also be manufactured using any of the manufacturing techniques or materials, previously described above with reference to the proximal component 306 and/or distal component 304 of the outer cutting member 302. In some examples, the outer cutting tube 352 may be made from extruded tubing, with additional finishing process performed to form any of the outer window 356, the cutting edge 354, and/or the first sloped surface 358 and/or the second sloped surface 368. In one such example, any of the cutting edge 354, the first sloped surface 358, or the second sloped surface 368 may be defined in the outer cutting tube 352 using a laser-cutting process.

In view of the above, the outer cutting member 302 may provide similar benefits and/or advantages to the tissue resection system 100, among other tissue resection devices, as the first cutting edge 326 and/or the first sloped surface 328 of the outer cutting member 302. More specifically, at least because a distal area (i.e. the cutting edge 354) of the outer window 324, and a distal end (i.e. the second cutting edge 336) of the inner cutting tube 112, may be sharpened to help cut or sever tissue, cut quality and precision, as well as cutting efficiency, may be enhanced over reciprocating cutting assemblies without two sharpened cutting surfaces.

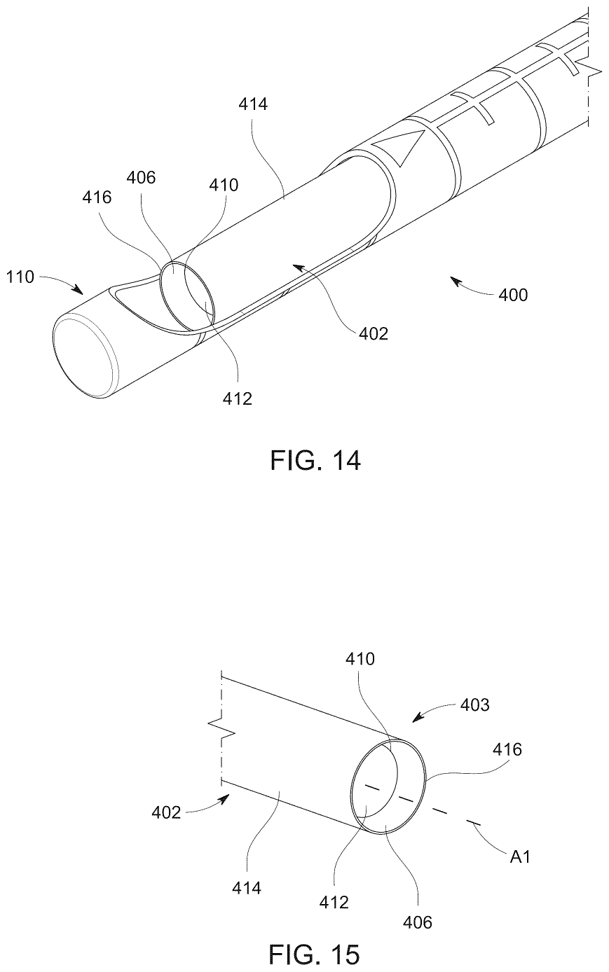

Finally, the tissue resection system 100 may include, as shown in FIGS. 14-15, a cutting assembly 400 including the outer cutting tube 110 and an inner cutting tube 402 defining an inverse cutting surface 406. However, in other examples, the cutting assembly 400 may include the outer cutting member 302. The inner cutting tube 402 may be similar to the inner cutting tube 112 previously discussed above, except in that, rather than including the second sloped surface 332 which tapers towards the central axis A1 in a distal direction, a distal end 403 of the inner cutting tube 402 includes an inverse cutting surface 406 which tapers towards the central axis A1 in a proximal direction, or in other words, tapers outwardly away from the central axis in a distal direction.

More specifically, the inverse cutting surface 406 may slope or taper towards the central axis A1, such as between an inner surface 410 defining a lumen 412 of the inner cutting tube 402, and an outer surface 414 of the inner cutting tube 402. Additionally, the inner cutting tube 402 includes a distal cutting edge 416. The distal cutting edge 416 may be a sharped surface adapted for cutting or severing tissue defined by an interface, intersection, or a meeting point where the inverse cutting surface 406 and the outer surface 414 of the inner cutting tube 402 meet. In some examples, the surface area of the distal cutting edge 416 and/or the inverse cutting surface 406 may be smooth or polished, or alternatively, the surface area of distal cutting edge 416 and/or the inverse cutting surface 406 may be serrated, such as by including a variety of different cutting projections and/or protrusions.

The inverse cutting surface 406 may also form various acute angles, in a proximal direction, relative to a single point or tangent along the central axis A1. In some examples, the inverse cutting surface 406 may form an angle within an inclusive range of about 5 degrees and about 50 degrees. In one specific example, the inverse cutting surface 406 may form an angle of about 15 degrees relative to the central axis A1. In this regard, it is also appreciated that the sharpness of the distal cutting edge 416 may be increased by reducing the angle that the inverse cutting surface 406 forms relative to a single point or tangent along the central axis A1. In some examples where the cutting assembly 400 includes the outer cutting member 302 or the outer cutting tube 352, it is also appreciated that the inverse cutting surface 406 and the first sloped surface 328, or the inverse cutting surface 406 and the first sloped surface 358, may be configured to form similar or different angles relative to one another.

The inner cutting tube 402 may also be manufactured using any of the techniques or materials previously described above with reference to the proximal component 306 and/or distal component 304 of the outer cutting member 302. In some examples, the inner cutting tube 402 may be made from extruded tubing, with additional finishing process performed to form the inverse cutting surface 406 and/or the distal cutting edge 416. In one such example, any of the cutting edge 354, the first sloped surface 358, or the second sloped surface 368 may be defined in the outer cutting tube 352 using a laser-cutting process.

In view of all the above, the distal cutting edge 416, by virtue of the inverse cutting surface 406, may possess an outer diameter or circumference that is similar, or identical, to an outer diameter or circumference defined by the outer surface 414 of the inner cutting tube 402. As may be appreciated, such an arrangement may enable the distal cutting edge 416 to, such as relative to the second cutting edge 336 of the inner cutting tube 112, be positioned much closer, in radial direction, to the outer cutting tube 110 at a location where tissue cutting or shearing action occurs. Thus, the inner cutting tube 402 may increase cutting efficiency and consistency at least by reducing tissue drag and/or deflection over typical or traditional inwardly tapered or chamfered cutting tubes, without significantly increasing manufacturing cost.

Although the invention has been described in terms of particular examples and applications, one of ordinary skill in the art, in light of this teaching, can generate additional examples and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A uterine tissue resection system comprising:
a housing comprising a handle;
a power source within the housing;
a motor within the housing and connected to the power source;
an outer cutting tube having a first lumen, a closed distal end, and a window through a sidewall thereof leading to the first lumen, the window being located proximal of the closed distal end;
an inner cutting tube received within the first lumen and having a second lumen extending between an open distal end and an open proximal end, wherein the open proximal end is connected to an external suction source thorough a swivel interface adapted to enable free rotation of the housing relative to the external suction source; and
a drive assembly located within the handle and connecting the inner cutting tube to a driveshaft of the motor such that when the motor receives power from the power source, rotation of the driveshaft causes the inner cutting tube to rotate and reciprocate concurrently.

2. The system of claim 1, wherein the swivel interface includes a first swivel connection fluidly coupling the external suction source to a suction tube within the housing.

3. The system of claim 2, wherein the first swivel connection includes a first rotatable connector including a first end comprising a proximal barb nipple and an opposite second end comprising a distal barb nipple.

4. The system of claim 3, further comprising a second swivel connection fluidly coupling the suction tube to the inner cutting tube; and wherein the second swivel connection includes a second rotatable connector including a first end comprising a proximal barb nipple and a second end adapted to receive a seal extending radially outward from a proximal end of the inner cutting tube.

5. The system of claim 1, further comprising a control board located within the housing, the control board adapted to regulate operation of the motor and discharging and/or charging of the power source.

6. The system of claim 5, wherein the drive assembly comprises:
an inner cutting tube received within the outer cutting tube, wherein the inner cutting tube is adjacent to the driveshaft and parallel thereto;
a first spur gear connected to the driveshaft;
a second spur gear surrounding the inner cutting tube and meshed with the first spur gear;
a worm gear positioned adjacent and meshed with a worm connected to the driveshaft such that rotation of the worm around a first axis results in rotation of the worm gear around a second axis; and
a crank attached to the worm gear and a sleeve encompassing a portion of the inner cutting tube.

7. The system of claim 6, wherein the second spur gear defines a longitudinal groove and the inner cutting tube includes a sleeve defining a longitudinal projection; wherein the longitudinal groove is configured to receive the longitudinal projection to enable the second spur gear to rotate the inner cutting tube as the inner cutting tube reciprocates proximally and distally within the second spur gear.

8. The system of claim 7, wherein the second axis is perpendicular to the first axis.

9. The system of claim 6, further comprising:
a power switch connected to the control board; and
an activation switch connected to the control board, wherein the control board is adapted prevent the motor from receiving power from the power source unless the power switch and the activation switch are closed.

10. The system of claim 9, further comprising a safety switch connected to the control board, wherein the control board is adapted to provide power from the power source to the motor, after the activation switch is released, until the safety switch opens.

11. The system of claim 10, wherein a lever of the safety switch remains in continuous contact with a surface of the worm gear such that the safety switch is open when the inner cutting tube is in an extended position.

12. The system of claim 11, wherein the surface is comprised of a semi-circular segment, which extends at least partially around a central axis of the worm gear, and inset segment; and wherein the safety switch is open when the lever is in contact with the inset segment.

13-20. (canceled)

21. The system of claim 1, wherein the closed distal end of the outer cutting tube comprises a distal component attached thereto.

22. The system of claim 21, wherein the distal component comprises a first cutting edge.

23. The system of claim 22, wherein the first cutting edge extends within an inclusive range of about 45 degrees and about 270 degrees around a central axis of the outer cutting tube.

24. The system of claim 23, wherein the distal component comprises an inner surface sized to snugly receive the open distal end of the inner cutting tube.

25. The system of claim 1, wherein the outer window includes a cutting edge.

26. The system of claim 25, wherein the cutting edge is located only at a distal end of the window.

27. The system of claim 25, wherein the cutting edge is comprised of a sharpened meeting region between a first sloped surface and an inner surface of the outer cutting tube.

28. The system of claim 1, wherein the inner cutting tube includes an inverse cutting surface.

29. The system of claim 28, wherein the inverse cutting surface tapers towards a central axis of the outer cutting tube in a proximal direction.

30. The system of claim 29, wherein the inverse cutting surface forms an angle within an inclusive range of about 5 degrees and about 50 degrees relative to the central axis of the outer cutting tube.
